(19) United States
(12) Patent Application Publication
(10) Pub. No.: US 2018/0243580 A1
(43) Pub. Date: Aug. 30, 2018

(54) Intelligent Light Therapy System

(71) Applicant: LEDskin B.V., 's-Hertogenbosch (NL)

(72) Inventors: Josephus Johannes Theodoor Louis Verhoeks, 's-Hertogenbosch (NL); Rick Wielens, 's-Hertogenbosch (NL); Henri Chrétien Marie Sprengers, 's-Hertogenbosch (NL)

(21) Appl. No.: 15/753,010

(22) PCT Filed: Jul. 21, 2016
(86) PCT No.: PCT/NL2016/050546

§ 371 Date: Feb. 15, 2018

(30) Foreign Application Priority Data
Aug. 19, 2015 (NL) 2015320
Oct. 23, 2015 (NL) 2015652

(51) Int. Cl. A61N 5/06; G16H 50/20; G16H 40/63

(52) U.S. Cl. CPC A61N 5/06; A61N 5/0621; G16H 50/20; G16H 40/63; A61N 2005/0663; A61N 2005/0645; A61N 2005/0652; A61N 2005/0653; A61N 2005/0632

(57) ABSTRACT

Light therapy systems for example for the ambulant treatment of hyperbilirubinemia. An intelligent wearable light module for ambulatory light therapy disposed in a wearable light module housing arranged to be worn on the body of a patient for illuminating, during use of the module, the skin of the patient with light of a wavelength corresponding to the light therapy.

### TECHNICAL FIELD

The present invention generally relates to the field of light therapy systems for example for ambulatory care of hyperbilirubinemia.

### BACKGROUND OF THE INVENTION

Light therapy, or also known as phototherapy or even heliotherapy, consists of exposure to a subject, i.e. a patient, of daylight or artificial light with a certain specific wavelength. Depending on the treatment and condition the light is administered for a predetermined amount of time, at a certain intensity and in often at a specific time of day, such as during night.

Amongst others, light therapy is applied for medical uses for the treatment of skin conditions such as psoriasis and acne vulgaris. However, light therapy is also a well-established medical therapy for the treatment of bilirubin defects such as neonatal jaundice. It is well known in the art that light with a specific wavelength between 450 to 500 nm has a positive effect on reduction of the bilirubin levels in the human body, and as such light therapy is considered an efficient therapy in reduction of bilirubin.

In the art two methods are know to apply light therapy for bilirubin reduction. The most common is the use of a high-powered fluorescent lighting, which is placed at some distance of the patient. When the patient is an infant, at the treatment of neonatal jaundice for example, this high-power fluorescent light is placed near or above a couveuse/isolette incubator such that the light rays are directed to the infant and due to the distance between the infant and light, a large part of the body of the infant is illuminated. During the light therapy the infant is either naked, or only wears a diaper. There is a serious risk of hydration and tissue damage such as radiation burn as a result of overexposure by the illuminated light. The infant also needs to be kept in the correct position to receive the light therapy. Often infants are fixed in one way or the other or even sedated to keep them in the correct position for the duration of the therapy. Since the high-power lights can cause damage to the eyes as well, the eyes of the infant need to be covered with patches. All in all these measures have a very negative effect on the comfort level of the patient. When the patient is an infant this is in particularly undesirable.

In an alternative method, use is made of optical fibres in which the light of a remote light source is coupled in the fibre and accordingly transported to the skin of the patient. This method solves some of the disadvantages of the other method, for example because the light is not directed to the eyes, which thus do not need to be covered. However, this alternative method with optical fibres has other disadvantages. The area of skin illuminated by a single optical fibre is very limited. Hence, a large amount of fibres is needed to achieve the desired radiation prescribed by the therapy. This results in a large bundle of fibres that all have to be connected, i.e. coupled, to a main unit wherein the light source(s) are located. This makes the system large, heavy and cumbersome, which also does not have a positive effect on the comfort level of the patient, and which is again in particular in case of infants undesirable.

Known light therapy devices that use either one of the methods described above have further limitations due to fact that a doctor, nurse or other medial personal has to monitor the influence of the light therapy on the patient on a regular basis. Conventional prior art light therapy systems are thus used in hospitals only, where the therapy can be performed under the surveillance of trained and qualified medical staff of the hospital. If the temperature of the skin starts to rise above a predetermined desired level, or the skin colour changes to a certain degree, the medical staff can act accordingly by changing the configuration of the therapy, for example by lowering the light intensity.

With the ever-increasing expenses of public healthcare, the need for ambulatory care increases as well. Moreover, from a patient's comfort point of view, undergoing light therapy outside of the hospital in the comfort of the home environment, in particular in the case of infant patients, is highly desirable as well. And medical specialists always want to monitor and get feedback from patients.

Other known light therapy devices focus on mainly curing the body by warmth using infrared radiation or by a combination of wavelengths for inflammation, edema and muscular stimulation. These applications however require other wavelengths ranges than being suitable for bilirubin treatments. This treatments have a short distance control by practitioners, whereas for reducing cost monitoring and control at longer distance will be required.

### SUMMARY OF THE INVENTION

Accordingly, it is an object of the present invention to provide a novel light therapy system in which at least some of the limitations and drawback of known light therapy systems have been obviated, in particular for baby patients in their first or second weeks after birth.

It is a further object of the present invention to provide a novel light therapy system with an increased comfort level for the patient.

It is yet another object of the present invention to provide a novel light therapy system which is remotely configurable for the therapy program, i.e.

condition treatment and/or to the individual patient.

In a first aspect of the invention there is provided an intelligent wearable light module for ambulatory light therapy, the light module being disposed in a wearable light module housing arranged to be worn on the body of a patient for illuminating, during use of the module, the skin of the patient with light of a wavelength corresponding to the light therapy, the light module comprising:
a substrate, comprising a plurality of light emitting diodes, LED's (which can be LED strips, separate LEDs or OLEDs), arranged in a light emission plane on an emission side of the wearable light module housing facing, during use, towards the skin of the patient;
a driver unit, arranged for driving the plurality of LED's (LED strips, separate LEDs or OLEDs) on the substrate;
a power supply, arranged for powering the light module;
a small low-power communication unit, arranged for remote communication with the light module; the light module further comprising:
a local data storage unit, arranged for locally storing data;
a processing unit, arranged for controlling the plurality of LED's (LED strips, separate LEDs or OLEDs) by control of the driver unit, and for logging data related to driving the driver unit, and for access to the local data storage and the data stored therein, by means of the small low-power communication unit.

By using LED's (either LED strips, separate LEDs or OLEDs) as light source, temperature induced, power requirements, and structural dimensions are significantly reduced specially to be used for young patients. This enables the light module to operate at low temperature such that direct skin contact does not cause problems. The low power requirements enable the use of a low weight battery and the small structural dimensions of the substrate with the LEDs especially OLEDs enables a small form factor, i.e. small structural dimensions for the housing of the light module. Accordingly, the light modules can be worn directly on the patient, which obviates the use of eye patches, and due to the low weight and form factor add to the comfort level of the patient. More than one module can be effectively used simultaneous, and all modules can communicate to an external connection unit for long distance control or with each other. Depending on the applicantion different wireless protocols can be used from low power till high mobile frequencies or wired.

Public health expenses are ever increasing. In view thereof, and in view of increasing comfort for the patient, ambulatory care is getting more and more important. By using a basic station the exposure to radiation for the patient can be kept very low and contact visa versa can be made remotely via internet or mobile network.

The invention is based on the insight that in order to be able to provide such ambulatory care, a light therapy device does not only need to be small in dimensions and easy to use, it needs to be able to provide feedback information on the progress of the therapy. Conventional systems know in the art are not able to provide such feedback. The feedback is in conventional systems fully up to in-situ evaluation of the current situation by the medical specialists, e.g. medical staff of a hospital. Preferably, the device is further arranged to be controlled remotely. Thus the medical specialist can monitor the therapy and progress of the therapy remotely, and can intervene the therapy by remote control.

To this end, a light therapy system according to the invention is comprises of at least one wearable light module. The light module is, as described, formed to such a degree that it can be worn on a body of a patient for illuminating, when the light module is activated, the skin of the patient with light of a wavelength corresponding to the therapy, such as blue light at circa 490 nm wavelength for hyper-bilirubin or any other wavelength or combination of wavelengths which fits the disease involved. The wearable light module comprises to this end a substrate with a few or several LEDs, depending on form factor and size of the substrate. The LED strips or separate LEDs are placed at a certain distance from each other in a certain pattern on the substrate such that the LEDs can emit light from an emission side of the wearable light module, which is facing towards the skin of the patient. For OLEDs the flexibility of the OLED itself is advantageous and does not need any separation as the complete OLED-foil can be used. The LED's (LED strips, separate LEDs or OLEDs) are thus either in direct contact with the skin, or at a small distance thereof, for example less than a few millimetres and for example by an intermediate shielding layer. Depending on the therapy needed, and the age or skin surface of the patient, the amount of light modules, the light frequency and light intensity can be defined on a patient per patient basis.

A light therapy system that is suitable for ambulatory care needs to have measures such that the need for medical staff evaluation is superfluous or at least reduced to such a level that it is safe for use outside the hospital, at home or a other private environment and hence away from medical staff. However the medical staff must be able to read out and control the device remotely from any place.

To this end the light therapy module according to the invention is light module that can communicate wirelessly and be controlled by a single stand alone control device, this being a dedicated control device, smartphone, tablet, personal computer, server or via another light module. The light module and/or control device is able to detect and monitor information, i.e. data by the system itself, which can be communicated to the medical staff via wireless and/or wired (e.g. internet) communication links including sent out alarms in case thresholds are exceeded. The information can comprise the time that the LEDs have been activated, the light intensity applied by the LEDs, the temperature induced by the LEDs and on the skin of the patient, battery levels, monitoring information of the wireless communication links between the light modules and the control device and/or the control device and a wide area network, e.g. the internet or a telephone connection, or patient data received from one of the sensors.

In this way the light therapy module is a source of information for scientific research and allows the use of trend analysis for the treatment application.

When using conventional light therapy systems, the medical staff of the hospital performs invasive lab tests on a regular basis. For example, the bilirubin levels in the blood of the infant (or adult) are tested once or two times a day by drawing blood and subjecting the blood to a chemical test in the laboratory of the hospital to determine the level of bilirubin. This is a labour intensive test, with high costs, which also requires lab work in the hospital.

In order to perform scientific research on the diseases and conditions for which the light therapy is used, e.g. neonatal jaundice, data of the therapy is needed, preferably large amounts thereof. From a combination of system data, such as temperatures, wavelengths, time durations, light intensity, and patient data such as skin colour change, skin temperature, heartbeat rate, etc. statistical analysis's can be performed. For example regression analysis can reveal certain relations between system parameters such as LED wavelengths used and skin colour or colour change trends, from which certain conclusions can be drawn which could add to further increase efficiency and effectiveness of the therapy and potential reduction of blood tests.

The light module according to the invention is therefor arranged to determine, e.g. measure and monitor, the system parameters, i.e. system data, and store these parameters to make them available for local or remote download such that it can be used in scientific research. Moreover, the system is further arranged to collect patient data by measuring patient parameters via one or more sensor disposed in the light modules or in a dedicated sensor module. This sensor determines for example the heartbeat rate of the patient, the skin temperature, the skin colour, etc. The sensor can be a sensor known in the art for determining temperature-on-skin, or to determine a heart rate. In order for the sensor to be able to determine skin colour, the sensor can be present in the light module in the form of a photocell, CCD image sensor, CMOS image sensor or a spectroscopic sensor for measuring irradiation reflectance. Additionally a sensor can measure the light intensity.

The light module is, preferably, power-self-sufficient by use of a battery. The module can however also be powered through a power cord. The battery can be either a replaceable battery disposed in the housing such that it can be removed very easily. Alternatively, a rechargeable battery can be used, wherein the housing is arranged for recharging the battery.

The LED's (either LED strips, separate LEDs or OLEDs) are driven by a LED driver unit, which preferably, is able to control activation of the LEDs, the light intensity of the LEDs, maximum power consumption, maximum temperature of the LEDs, etc. The values for the wearable light module can be set and controlled by the housing. Autonomous or remote controlled operation can be included.

The wearable light module further comprises a small low-power wireless communication functionality and/or unit. This way, the light module can be controlled through a dedicated control device, a mobile dedicated control device, an software application running on a communication device such as a tablet or smartphone, or through a remote located personal computer or server. If for any reason no wireless communication is possible or direct external battery supply is wanted (e.g. no internal battery modules) the light units can be connected to each other and communication can be done via the wired connection, which can also be used for the external battery supply.

The light module further comprises a processing unit such as a general-purpose processor, or a dedicated processor. The processing unit is arranged for wireless or wired control of at least one on the skin wearable light module via remote control, such as a dedicated control device, a mobile dedicated control device, an application running on a communication device such as a tablet or smartphone, or through a remote located personal computer or server.

Since different types of wearable light module can exist, having different form factors, wavelengths emitted, etc., each of the wearable light modules can comprises a unique identification value. With this unique identification value the processing unit is able to identify each wearable light module and determine, either from the wearable light module itself or from a local or remote storage, the properties of the wearable light module, e.g. the type, dimensions, wavelength used, etc.

Accordingly, the light therapy module according to the present invention is able to apply different types of therapies to all sorts of patients such by using a single control unit, and one or more wearable light modules in a configuration dedicated for the patient and therapy.

In an example, the light module further comprising:
at least one sensor unit disposed in on the skin wearable light module housing and comprising at least one sensor for determining patient data and transmitting the patient and/or system data to the processing unit for storage thereof in the local data storage unit.

In an example, the processing unit is arranged, by means of the small low-power communication unit, for communication with at least one sensor unit disposed in a separate wearable sensor module housing, for receiving patient data from the sensor module and for storage thereof in the local data storage unit.

As indicated, the light module is at least arranged to determine and store, in the local storage unit, data relating to the driving of the LEDs, for example runtime of the LEDs, remaining life-time, light intensity, power consumption, temperature, etc. The light module can however also comprise a sensor unit, or multiple sensor units and/or separate external sensor units with low-power wireless connection to the light module in case sensor data is to be retrieved on places of the skin not covered by the light module. These sensor units comprise one or more sensors that can determine patient data and provide the data to the processing unit for storage thereof in the storage unit or light module. Accordingly, both the data relating to the driving of the LEDs, i.e. system data, and patient data can be made available through the communication unit such that is can be accessed/downloaded remote, or locally.

In an example, the processing unit is arranged for determining configuration variables of the wearable light module and/or separate sensor units, and wherein the configuration variables in particular comprise any one or more one of the group consisting of: amount of LEDs on the substrate, type of LED, distribution of the LEDs in the light emission plane, duty cycle of the LEDs, power consumption of the plurality of LEDs, temperature of the wearable light module, power level of the battery, status of a communication link between the communication units of the wearable light module and the control device, emitted wavelength of the plurality of LEDs, type of LEDs on the substrate or stand alone for OLEDs, dimensions of the wearable light module. This includes the checking of the availability and the connection of external sensor units for one or more sensors and reading out the data via a low-power wireless link.

In an example, a light therapy system can comprise two or more light modules, for example each having a different configuration. The processing unit of the modules can recognise from the identification value their own configuration variables, but through the communication unit, also those of the other modules or sensors in the system. The system can also be comprised of a control device, such as a (local) dedicated control device, an application running on a communication device such as a tablet or smartphone, or a (local or remote) personal computer or server.

The system can preferably be arranged to handle, control and connect with a plurality of light modules, for example, two, three, four, five, six, eight, ten, or even up to 32 modules. These modules could for example be all of the same type, thus same amount and type of LEDs, power consumption, wavelengths, etc. These could also be a combination of several different types of modules having different configurations or different LED's such as separate LEDs or OLEDs. As such, the system can be build-up of modules in accordance with the specification as desired by the type of therapy the patient has to undergo, and/or the amount of modules needed to cover sufficient skin and be able to illuminate the skin with an amount of lumen needed for the therapy and according to the body size of the patient, i.e. age, weight and height.

In an example, the power supply comprises a battery, and wherein the battery is in particular arranged to be charged by a flexible connection such as a foil or flexible wire.

The power supply of the light module could be a power connector for connecting a (low voltage) power cord thereto, a power converter for converting from a conventional power line, or a battery. The battery or power connector could in particular be connected via a flexible foil or flexible wire.

In an example, the small low-power communication unit is arranged for wired communication, in particular via a flexible connection such as a foil or flexible wire.

In an example, the small low-power communication unit is arranged for low energy emission wireless communication, and in particular for wireless mesh network or near field communication, more in particular a Zigbee network or MyriaNed. In particular low energy levels in the wireless communication implemented in the embodiments of the invention is desirable as the modular wearable light modules are in particular suitable or intended to be used with new born or infants. With these patients any exposure to higher energy levels should be avoided.

The light modules can be disposed in a star type network topology wherein the light modules are only connected with the central controlling light module, or dedicated control device. As an alternative, the light modules could also communicate according to a mesh type network wherein the light modules can also communicate with each other and/or retrieve data from the external sensors. The advantage thereof is that intermediate light modules can act as a proxy for control signals sent from remote nodes within the network. This reduces radio transmission power consumption, which lowers the power supply, e.g. battery requirements, and thus increases battery run-time and/or battery weight. Moreover, from an EMC point of view, in particular in hospitals and the like, it is also advantageous to transmit with very low power. Examples of the mesh network communication types that are applicable are Zigbee or MyriaNed and also other wired or wireless mesh network. The skilled person will appreciate which other networks are applicable.

In an example, the small low-power communication unit is arranged for communicating via a base unit over a wide area network such as the internet or a public (mobile) telephone network, and wherein the communicating over the wide area network is performed over a point-to-point secure tunnel.

Preferably, the light modules are wirelessly or wired interconnected, and to the other end also connectable to a wide area network such as a public telephone network, either hard-wired, mobile or both, or to an internet connection, also either hard-wired, wireless or both. When connectable to more than one communication channel, this has the advantage that the system is more redundant and thus reliable. If one communication channel is not functioning, then there is always a backup/fall-back option. Preferably, especially when communicating over a public network such as an internet connection, the communication is performed in a secure manner, for example via a secure sockets layer, SSL tunnel, e.g. a SSL Virtual Private Network, VPN, tunnel.

In an example, the housing of the light module is composed of a soft material, in particular a material having a Shore 00 hardness between 30 and 90, preferably between 35 and 70, more preferably between 40 and 60.

Since the light modules are wearable modules, these can be placed either directly on the skin under the clothing, or in a garment in close contact with the skin. In either case, the housing of the modules is preferably produced from a soft, skin friendly material with a low Shore 00 hardness such that wearing the modules does not have a negative effect on the comfort level of the patient.

In an example, the housing of the light module is composed of a flexible material for following the contours of the skin, in particular a material having a Young's modulus less than 2 GPa, preferably less than 1 GPa, more preferably less than 0,1 GPa and even more preferably, less than 0,01 GPa.

The material used can be soft, but is preferably also flexible to such a degree that it can follow the contours of the skin and by which the distance between the LEDs and the skin is minimal over the full surface area of the light module, this has the advantage that it not only adds to the comfort level of a light module such that it can worn as clothing, it also enable the use of low power LEDs or OLEDs, which induce less heat and have smaller battery requirements.

In an example, the processing unit is arranged to receive a therapeutic program selection, and wherein the driver unit is controlled by the processing unit in accordance with the therapeutic program, and wherein the selection.

The processing unit is preferably arranged to execute a plurality of different therapeutic programs. These programmes are stored in a storage of a control device or one or more of the modules, and the processing unit thereof is able to configure the light modules in accordance therewith. Examples of configuration parameters are, time duration of the therapy, time duration of individual modules being activated, light intensity, light pattern executed (continuous, sine-, triangular-, shark tooth-, block-shaped wave pattern, etc.), etc. Any user, or only restricted users such as medical staff via a username and password combination, can select a therapy to be executed from the list of therapy programs available in the control device. This selection can be performed locally on the control device directly, for example via a Graphical User Interface display, and/or via a remote communication channel such as a SSL VNP tunnel over the internet and/or via a local wireless control device, such as a dedicated control device or a general control device running a specific application, for example on a mobile (smart) phone, a tablet or the like.

An advantage of the ability of executing a therapeutic program and a light therapy system that is arranged for communication over a wide area network is that such a system is particularly suitable for publishing data of the therapeutic program, and the progress of the patient within the program on social media. For example, parents can be provided access and control to publish data, i.e. program variables, sensor data, etc. on social media such as Twitter, Facebook etc.

In an example, the processing unit is arranged to receive a configuration variable setting of a therapeutic program, and wherein the driver unit is controlled by the processing unit in accordance with the configuration setting.

The configuration variables are preferably stored such that these can be used as scientific data in a scientific research on the diseases and conditions for which the light therapy system is used. By storing not only the configuration variables, i.e. parameters of the system but also monitoring and storing, i.e. logging, system data such as time of use, battery information, etc. relevant data can be obtained for determining relations between parameters of the system and the diseases and conditions for which they are used. These can help to increase knowledge about the diseases and conditions and to improve the system itself. The data can be made available via the communication unit, for example for local download by an USB connection, and/or for remote downloading from an internal storage location that has been made available in a secure manner over an internet connection.

In an example, the patient data determined by the at least one sensor comprises any one or more of the group consisting of body temperature, skin temperature, skin colour, heartbeat rate and blood pressure.

The light modules comprise at least one sensor, or are arranged to receive patient data from one or more dedicated sensors. With the sensors patient data is determined such that it can be stored and preferably pre-processed by the processing unit of a control device or one of the light modules. With pre-processing is meant that the data is for example checked on reliability, whether or not it exceeds certain thresholds that indicate incorrect measurement or a system failure for example. Pre-processing could also encompass making the data anonymous by removing certain data or relations between data that could reveal personal information. The patent data, either pre-processed or in raw form, can then be made available for remote or local download in accordance with the system data as described above.

The sensor or sensors (in the modular wearable light module or implemented in a separate wearable sensor module placed elsewhere on the patient's body) can determine preferably one or more relevant patient data such as body temperature, skin temperature, skin colour, heart rate, blood pressure, skin moisture levels, traveling distance, blood oxygen levels, blood sugar levels, etc. but also system data such as light intensity. The skilled person will appreciate that several different types of patient data can be determined by such sensors

In an example, the determined patient data are compared by the processing unit with at least one, and preferably multiple, threshold value, for signalling an alarm upon exceeding the threshold value.

The system preferably comprises configuration variable thresholds, preferably multiple. This has the advantage that the system is more safe to use since it actively monitors relevant configuration variables or parameters of the device such as time of use, i.e. time duration of the device, light intensity, temperature, battery level, etc. Whenever a threshold is exceeded, or one of the intermediate thresholds is exceeded, the processing unit can take necessary action, such as sending an alarm to medical staff, activate an alarm light on the control device, activate a alarm audio signal, send a phone signal (e.g. SMS or call) to the user, etc. Preferably the system is arranged not only to inform and alarms upon determining that a variable exceeds a threshold, but is also able to alter relevant configuration variables such that the threshold is not exceeded anymore, for example by shutting down certain modules, lowering light intensity, etc. Altering these configuration variables can be done distantly by the medical staff via internet and/or wireless in a secured way, avoiding unwanted changes.

In an example, the light module further comprises a thermal interface arranged for heat dissipation from the plurality of LEDs to the environment.

To add to the comfort of the patient, the heat dissipated by all LEDs or OLEDs is preferably removed from the side of the light modules that face the patient, and dissipated toward the backside thereof. This way the modules do not exceed the patient's body temperature and thus do not increase skin temperature.

In a second aspect, there is provided an intelligent wearable sensor module for ambulatory light therapy, the sensor module being disposed in a wearable sensor module housing arranged to be worn on the body of a patient and comprising:
a power supply, arranged for powering the sensor module;
at least one sensor for determining, during use of the module, patient data;
a small low-power wireless communication unit for transmitting the patient data to the processing unit of an intelligent wearable light module according to any of the previous descriptions.

In a third aspect, there is provided an intelligent control device for ambulatory light therapy, comprising:
a power supply, arranged for powering the control device;
a small low-power communication unit, arranged for remote communication with at least one modular wearable light module according to any of the previous claims descriptions;
a processing unit, arranged for control of the at least one modular wearable light module through the small low-power communication unit.

In a fourth aspect, there provided, an intelligent control device for ambulatory light therapy according to the previous description, wherein the small low-power communication unit is arranged for communicating over a wide area network such as the internet or a public (mobile) telephone network, and wherein the communicating over the wide area network is performed over a point-to-point secure tunnel.

In an example, the power supply is arranged for powering a power supply of at least one modular wearable light module according to any of the previous descriptions, and in particular for charging the battery thereof.

The power supply of the control device can be provided with a power line connection. Through this power line connection and power supply the power supplies of the light modules of the therapy system can be powered. If the light modules comprise a battery, the battery can also be charged through the power supply of the control device. The charging can be done in a conventional manner wherein the light modules can be wired to the control device, or by induction charging on a specific induction pad of the control device. Moreover, the modules and/or control device could also be provided with solar cells to charge the modules via solar power. The control device could however also be formed in a way that it can receive a light module in a particular part of the housing with a shape mating the light module, such that it can be charged there by connecting with for example a power connector or other connection.

In a fifth aspect, there is provided an intelligent wearable ambulatory light therapy system, comprising:
one or more intelligent wearable light module according to any of the previous descriptions.

In an example, the light therapy system further comprises:
one or more Intelligent wearable sensor module according to previous descriptions.

In an example, the light therapy system further comprises:
a control device according to any of the previous descriptions, arranged for controlling one or more intelligent wearable light module according to any of the previous descriptions.

In the present invention wherever a control device is introduced, it is to be understood as a dedicated control device that is arranged to be carried on a body of the patient, in accordance with the light modules, or as a dedicated device that can be placed on a table for example, or a non-dedicated (mobile) communication device such as a smartphone, tablet, etc. with a dedicated software application running thereon. It is also to be interpreted as a personal computer, laptop or server, either local on-site, or located at a remote location such as a hospital or the like.

The above-mentioned and other features and advantages of the invention will be best understood from the following description referring to the attached drawings. In the drawings, like reference numerals denote identical parts or parts performing an identical or comparable function or operation

### BRIEF DESCRIPTION OF THE DRAWINGS

FIG. 1 shows a schematic illustrating a light therapy system according to an embodiment of the invention;

FIG. 2 shows a schematic illustration of a light module of a first type in a first configuration according to an embodiment of the invention;

FIG. 3 shows a schematic illustration of a light module of a second type in a second configuration according to an embodiment of the invention;

### DETAILED DESCRIPTION OF THE DRAWINGS

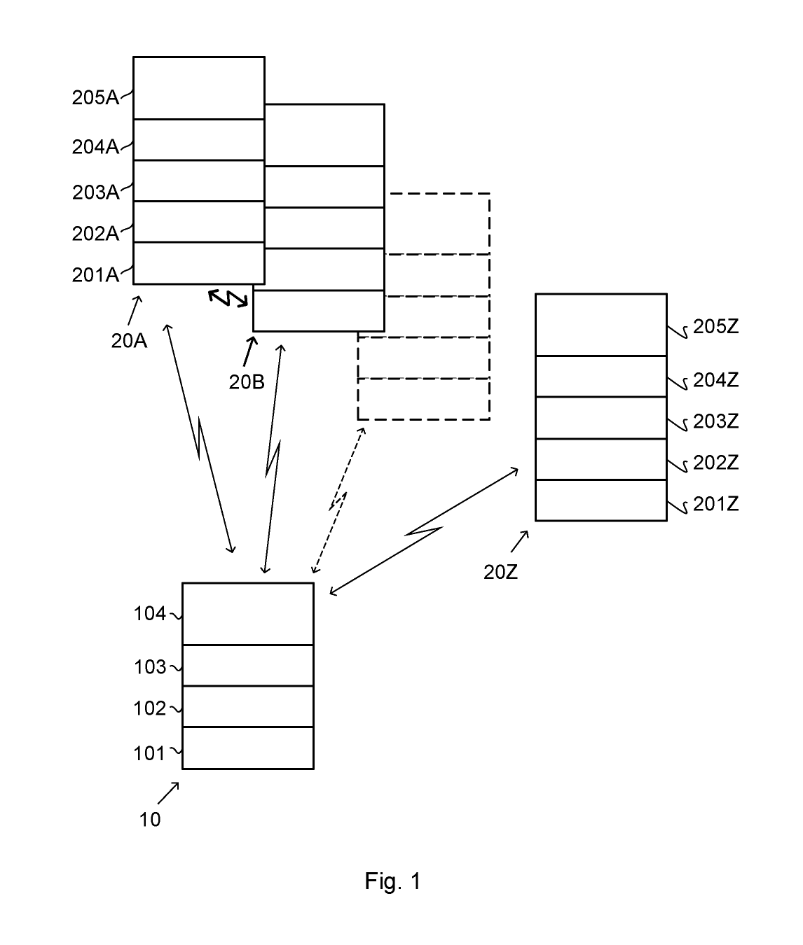

FIG. 1 is a schematic illustration of a light therapy system according to a first example of the invention. The light therapy system is an intelligent wearable ambulatory light therapy system. This means that the system can be used outside the walls of the hospital or professional care in a home environment, such in a save manner. The system comprises two different types of devices, i.e. the control device 10 and one or more light modules 20A, 20B, 20 . . . , 20Z. Each device 10, 20A-Z has its own separate housing. Preferably all parts 10, 20A-Z communicate with each other wirelessly, via a low-power mesh network, and/or wired as requested.

Each wearable light module is arranged to be worn under the clothing on a body of a patient in such a way that it can for illuminating, during the use of the system, the skin of said patient with light of a certain wavelength and intensity that corresponds to the therapy applied. This could be for example be a blue light in a wavelength around 490 nm, which is in particular suitable for bilirubin defects such as neonatal jaundice. The light modules can be attached in a special garment in which each module is fixed to the inside of the garment. The position in the garment can be defined by a pocket at the inside of the garment, which is able to receive the light module therein. The garment can however also be attached by Velcro or other suitable fixation means. During therapy the garment can then be worn by the patient with a high level of comfort. To this end the wearable light module is also able to resist water and dust, and has for example a liquid ingress protection level 3, 4, 5, or even up to level 6 and level 1, 2, 3, 4, 5 or even level 6 solid particle protection, both according to standard international protection marking, IP code enabling sterilisation and/or disinfection

Each housing of the light module 20A-Z comprises several parts or units. Each module has a battery unit 201A that is disposed within the housing. The battery can be a removable battery or, preferably, a fixed non-removable rechargeable battery that can be recharged via a charge interface 102 on the control device 10 or via on the light module integrated solar cells.

The light module comprises a plurality of Light Emitting Diodes, LED's (LED strips, separate LEDs or OLEDs). One module can comprise any number of separate LEDs e.g. 2, 4, 8, 16, 32, 64, 128, 256 or even more. The LEDs are disposed on a substrate, which provide fixation of the LEDs and provide electrical circuitry to drive the LEDs. In case of OLEDs the size of the foil can be chosen. As such, each module further comprises a LED driver 202A-Z. The LED driver drives the plurality of LED's (LED strips, separate LEDs or OLEDs) and is arranged to pulse the LEDs on an off by applying power periodically or intermittently.

The driver 202A-Z can be arranged for pulse-width-modulation and duty cycle of the LEDs, and/or driving said LEDs via a controlled DC-current.

The configuration of the light modules can differ. For example, one module 20A could contain 65 separate LEDs with a wavelength around 490 nm and another module 20B could contain 120 LEDs with a wavelength around 450 nm, and another module OLDs, etc. The number or area of LEDs to be activated is according the stored therapy program. Moreover, each module could have different dimensions. As such, modules could be numbered or coded from which number one can determine the configuration. For example PJ110XS could be a code for a light module configured especially for prenatal jaundice therapy, consisting of 110 LED, in an extra small form factor suitable for infants. Where PJO4×5 could be the code for an OLED of 4 by 5 cm.

Light modules are further arranged to determine patient data via a sensor unit 203A-Z. The sensor unit can be a sensor for measuring patient parameters like the heartbeat of the patient, the skin temperature, the skin colour, etc. or system data, such as the light intensity. The sensor can be a sensor known in the art for determining temperature-on-skin, or to determine a heartbeat. In order for the sensor to be able to determine skin colour, the sensor can be present in the light module in the form of a photo cell, CCD image sensor, CMOS image sensor or a spectroscopic sensor or implemented in a separate sensor unit for measuring irradiation reflectance and calculate changes.

The light modules further comprise a wireless communication unit 204A-Z. With the wireless communication unit the module is arranged for wireless communication with at least the control device and preferably with other modules as well. The communication is performed according to a short-range communication protocol with low energy emission levels, such as Bluetooth, Wi-Fi, or mesh type wireless personal area network such as Zigbee, MyriaNed network or other IEEE 802.15 conforming protocol. Herewith new born or infants are not subjected to too high energy levels, Alterably the communication is done via a flexible wired connection.

The light modules comprise a dedicated local storage for local buffering and a register for storing a unique identification value. This unique identification value could alternatively also be stored within the wireless communication module 204A-Z for example in the form of a unique network address. From the unique identification value the control device can determine for example therapy program, amount and type of LEDs, and form factor. This way the control device 10 is aware of the capabilities of the module and can execute a therapeutic program in accordance therewith.

The control device 10 comprises to this end a communication unit 104 to communicate with all modules via communication units 204A-Z thereof. In accordance with the communication protocol supported by these communication units 204A-Z, the communication unit of the control device is also able to communicate, e.g. Zigbee, MyriaNed, Bluetooth, WiFi, 3G, 4G etc. or even wired.

The light modules and/or control device comprise a local storage for storing system data and/or patient data. A further advantage of the local storage is that this provides a buffer to prevent data loss when the modules and/or control device suffer from connection loss. The buffer is thus in accordance with a certain preferred maximum time duration in which the connection can be lost.

The control device 10 further comprises a power unit 101 that powers the control device and can consist of a power line connector and/or internal battery. Optionally the control device may also power the separate light modules (not shown).

The batteries of the light modules can also be charged via a charge unit or interface 102. This can be performed by plugging in a cable or attaching the light module to the charge unit 102, by putting the module 20A-Z in a specific receiving part of the control device such that it can connect and charge the battery, or even via an inductive charging unit.

The control device is furthermore comprised of a processing unit 103. The processing unit is arranged to control all parts of the control device itself e.g. the communication with the light modules via the communication unit 104. The processing unit 103 has furthermore the purpose of storing all relevant data obtained within the system itself, i.e. system parameters or system variables such as battery information and run time. But the processing unit 103 is also able to store all relevant patient data obtained via sensor unit(s) 203 of the light modules. In an advanced embodiment the processing unit can make all patient data and system data for local or remote download. The local download can be performed via for example an USB interface (not shown), the remote download can be performed via a secure internet connection for example. To this end the communication unit 104 of the control device is arranged for communication over the internet, and/or mobile telecommunication network, and/or wireless telecommunication network.

The processing unit 103 of the control device can preferable also be arrange to transmit and receive configuration parameters. This way the control device can be monitored and control remotely, for example by a central medical centre wherein medical staff can live monitor the therapy or perform pre-, or post-therapy checks. Through a secure app (software application on a mobile device) the authorised doctor can monitor and control the system with a smartphone or PC. Moreover, the light therapy system, in every embodiment of the invention as described, can preferably not only be arranged to provide live monitoring for the patient by medical specialists, but is also arranged to be controlled by the medical specialist from a remote location, for example to increase or decrease light intensity, duration, etc. or to abort the therapy and de-activate the module(s). In an advanced embodiment, the system can also be arranged to operate autonomously, hence wherein the system can change configuration such as light intensity, duration, etc. or even de-activate upon exceeding a predefined threshold value. In yet an even more advanced embodiment the system can be arranged to set the thresholds autonomously or change the thresholds when considered necessary and/or safe.

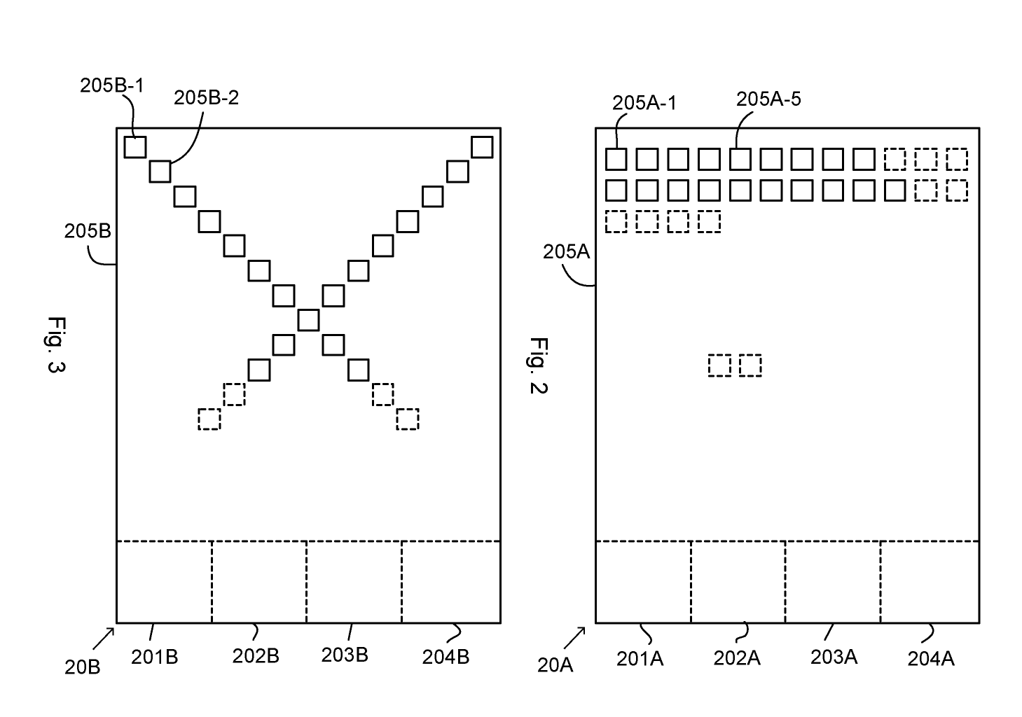

In FIGS. 2 and 3 illustrations are shown of different types of configurations of the light therapy modules. In FIG. 2 a light therapy module 20A is shown that can be connected to the control device of the system as indicated in FIG. 1. To this end the module also comprises a communication unit 204A, which is powered by a battery 201A or by a power connection to the processing unit (as this is optional it is not shown). The module also has one sensor unit 203A, (comprising one or more sensors). The module 20A of FIG. 2 comprises a large amount of individual LEDs 205A-1 . . . 205A-5 . . . 205A- . . . The individual LEDs are, by way of example, only partly shown. The substrate to which the LEDs are fixed, can for example comprises a 10×20 LED matrix having 10 rows and 20 columns of LEDs, which can be driven simultaneously, on an individual basis or on a row or column basis. FIG. 3 shows a different configuration wherein the LEDs are disposed on the substrate in an X character layout. Not shown is where the whole area is covered by an OLED foil.

The skilled person will appreciate that several other configurations and layouts are also applicable, and although not described in detail these also fall within the scope of the appended claims.

1. A modular wearable light module for ambulatory light therapy, the light module being disposed in a wearable light module housing arranged to be worn under the clothing on the body of a patient for illuminating, during use of the module, the skin of the patient with light of a wavelength corresponding to the light therapy, the light module comprising:
an OLED foil or substrate, comprising a plurality of light emitting diodes (LEDs) arranged in a light emission plane on an emission side of the wearable light module housing facing, during use, towards the skin of the patient;
a driver unit, arranged for driving the plurality of LEDs) on the substrate;
a power supply, arranged for powering the light module;
a low-power wireless communication unit, arranged for remote communication with at least the light module;
a local data storage unit, arranged for locally storing data; and
a processing unit, arranged for controlling the plurality of LEDs by control of the driver unit, and for logging data related to driving the driver unit, and for access to the local data storage and the data stored therein, by means of the low-power wireless communication unit.

2. The modular wearable light module for ambulatory light therapy according to claim 1, further comprising:
at least one sensor unit disposed in the wearable light module housing and comprising at least one sensor for determining patient data and/or system data and transmitting the data to the processing unit for storage thereof in the local data storage unit.

3. The modular wearable light module for ambulatory light therapy according to claim 1, wherein the processing unit is arranged, by means of the low-power wireless communication unit, for wireless communication with at least one sensor unit disposed in a separate wearable sensor module housing, for receiving the data from the sensor module and for storage thereof in the local data storage unit of the light unit.

4. The modular wearable light module for ambulatory light therapy according to claim 1, wherein the processing unit is arranged for determining one or more configuration variables of the wearable light module; and
wherein the configuration variables are selected from the group consisting of: amount of LEDs on the substrate, distribution of the LEDs in the light emission plane, duty cycle of the LEDs, power consumption of the plurality of LEDs, temperature of the wearable light module, power level of the power supply, status of a communication link between the communication units of the wearable light module and the control device, emitted wavelength of the plurality of LEDs, type of LEDs on the substrate, and dimensions of the wearable light module.

5. The modular wearable light module for ambulatory light therapy according to claim 1, wherein the OLED foil or substrate comprises OLEDs.

6. The modular wearable light module for ambulatory light therapy according to claim 1, wherein the power supply comprises a battery; and
wherein the battery is arranged to be charged by a flexible connection.

7. The modular wearable light module for ambulatory light therapy according to claim 1, wherein the low-power communication unit is arranged for wired communication.

8. The modular wearable light module for ambulatory light therapy according to claim 1, wherein the low-power communication unit is arranged for wireless communication.

9. The modular wearable light module for ambulatory light therapy according to claim 1, wherein the low-power communication unit is arranged for communicating over a wide area network;
and wherein the communicating over the wide area network is performed over a point-to-point secure tunnel.

10. The modular wearable light module for ambulatory light therapy according to claim 1, wherein the housing of the light module is composed of a a material having a Shore 00 hardness between 30 and 90.

11. The modular wearable light module for ambulatory light therapy according to claim 1, wherein the housing of the light module is composed of a flexible material having a Young's modulus less than 2 GPa.

12. The modular wearable light module for ambulatory light therapy according to claim 1, wherein the processing unit is arranged to receive a therapeutic program selection; and
wherein the driver unit is controlled by the processing unit in accordance with the therapeutic program.

13. The modular wearable light module for ambulatory light therapy according to claim 12, wherein the processing unit is arranged to receive a configuration variable setting of the therapeutic program; and
wherein the driver unit is controlled by the processing unit in accordance with the configuration variable setting.

14. The modular wearable light module for ambulatory light therapy according to claim 2, wherein the patient data determined by the at least one sensor is selected from the group consisting of body temperature, skin temperature, skin color, heartbeat rate and blood pressure.

15. The modular wearable light module for ambulatory light therapy according to claim 14, wherein the determined patient data are compared by the processing unit with at least one threshold value for signaling an alarm upon exceeding the threshold value.

16. The modular wearable light module for ambulatory light therapy according to claim 1 further comprising a thermal interface arranged for heat dissipation from the plurality of LEDs to the environment.

17. The modular wearable light module for ambulatory light therapy according to claim 1 further comprising a unique identification value stored on the light module, for identification of the light module and for remote determining configuration variables of the wearable light module; and
wherein the configuration variables are selected from the group consisting of: amount of LEDs on the substrate, distribution of the LEDs in the light emission plane, duty cycle of the LEDs, power consumption of the plurality of LEDs, temperature of the wearable light module, power level of the power supply, status of a communication link between the communication units of the wearable light module and the control device, emitted wavelength of the plurality of LEDs, type of LEDs on the substrate, and dimensions of the wearable light module.

18. (canceled)

19. An intelligent wearable sensor module for ambulatory light therapy, the sensor module being disposed in a wearable sensor module housing arranged to be worn on the body of a patient and comprising:
a power supply, arranged for powering the sensor module;
at least one sensor for determining, during use of the module, patient data; and
a low-power communication unit for transmitting the patient data to the processing unit of an intelligent wearable light module according to claim 1.

20. An intelligent control device for ambulatory light therapy, comprising:
a power supply, arranged for powering the control device;
a low-power communication unit, arranged for remote communication with at least one modular wearable light module according to claim 1,
a processing unit, arranged for control of the at least one modular wearable light module through the small low-power communication unit.

21. The intelligent control device for ambulatory light therapy according to claim 20, wherein the power supply is arranged for powering a power supply of at least one modular wearable light module according to claim 1.

22-24. (canceled)

25. The modular wearable light module for ambulatory light therapy according to claim 1, wherein the power supply comprises a battery;
wherein the battery is arranged to be charged by a one or more of a foil, by solar power, and induction;
wherein the low-power communication unit is arranged for one of:
wired communication via a flexible connection selected from the group consisting of a foil and a flexible wire;
wireless communication selected from the group consisting of a wireless mesh network, a Zigbee network and MyriaNed; or
communicating over a wide area network selected group the group consisting of the internet and a public (mobile) telephone network.

26. The modular wearable light module for ambulatory light therapy according to claim 1, wherein the housing of the light module is composed of a a material having a Shore 00 hardness between 40 and 60.

27. The modular wearable light module for ambulatory light therapy according to claim 1, wherein the housing of the light module is composed of a flexible material having a Young's modulus less than 0.01 GPa.
